(19) United States
(12) Patent Application Publication
(10) Pub. No.: US 2026/0071968 A1
(43) Pub. Date: Mar. 12, 2026

(54) NEW METHOD FOR DETECTING FOUR SULFUR-CONTAINING PESTICIDES BASED ON GOLD AND SILVER NANO-MIMETIC ENZYME MATERIALS AND APPLICATION THEREOF

(71) Applicant: South-Central Minzu University, Wuhan City (CN)

(72) Inventors: Haiyan FU, Wuhan City (CN); Xueqing Zeng, Yichang City (CN); Hengye Chen, Wuhan City (CN); Yuanbin She, Beijing (CN); Wanjun Long, Yongzhou City (CN); Wei Lan, Wuhan City (CN)

(21) Appl. No.: 19/304,736

(22) Filed: Aug. 20, 2025

(30) Foreign Application Priority Data
Sep. 9, 2024 (CN) 202411258697.2

(51) Int. Cl. G01N 21/78; C01G 7/00; G01N 21/31; G01N 33/02

(52) U.S. Cl. CPC G01N 21/78; C01G 7/00; G01N 21/31; G01N 33/02; C01P 2004/64

(57) ABSTRACT

A new colorimetric detection method for sulfur-containing pesticides with nano-mimetic enzyme is provided. The synthesized gold-silver bimetallic nanocluster (Au—Ag NCs) has quasi-peroxidase activity and catalyze hydrogen peroxide to oxidize 3,3′,5,5′-tetramethylbenzidine (TMB) to form blue products under acidic conditions. Based on the electrostatic interaction between Au—Ag NCs and four sulfur-containing pesticides, the exposed Au and Ag elements further combine with S in pesticide molecules to form bonds, and Au—Ag NCs loses the quasi-peroxidase activity and blocks the catalytic oxidation process.

### CROSS-REFERENCE TO RELATED APPLICATIONS

This application claims priority to Chinese Patent Application No. 202411258697.2, filed on Sep. 9, 2024, the contents of which are hereby incorporated by reference.

### TECHNICAL FIELD

The disclosure belongs to the technical field of nano-mimetic enzyme material preparation and chemical analysis detection, and particularly relates to a method for controllably preparing a nano-quasi-peroxidase colorimetric sensor and detecting four sulfur-containing pesticides with high sensitivity.

### BACKGROUND

Four sulfur-containing pesticides—thiophanate-methyl, cartap, dimethoate and temephos—are commonly used as fungicides and insecticides in the process of crop production and planting. The four sulfur-containing pesticides have the characteristics of systemic activity, contact toxicity, broad-spectrum efficacy and high toxicity, and may quickly prevent and treat pathogenic nematodes, spider mites, mosquitoes and moths, jumping lice and other diseases and insect pests in rice, fruit trees, vegetables and forests, and may effectively improve crop yield and quality. However, people's long-term intake of food with excessive pesticide residues has potential risks of teratogenesis, carcinogenesis and mutation, and even acute/chronic poisoning, which seriously harms human health. In order to monitor the residue of sulfur-containing pesticides in food samples, it is of great significance to develop a rapid detection method of sulfur-containing pesticides with high specificity and sensitivity.

Conventional detection techniques, including chromatography, spectroscopy and enzyme inhibition, have made significant progress in the field of pesticide residue detection, but the conventional detection techniques often have some shortcomings, such as complicated operation, time-consuming detection and high cost, so it is still worth further exploration to develop a new sensing method with convenient operation, sensitivity and portability and low cost. Nano-mimetic enzyme colorimetric sensing technology has the advantages of high sensitivity, high selectivity, short response time and simple operating conditions, and represents a novel approach for detecting pesticide residues. Nano-quasi-peroxidase has attracted extensive attention because of its simple preparation process and clear color reaction mechanism. Compared with natural peroxidase, nano-quasi-peroxidase has better thermal stability and anti-interference, and may effectively improve the sensitivity and specificity of detecting pesticide residues. Based on the excellent characteristics of nano-quasi-peroxidase, the disclosure provides a new method for quantitatively detecting four sulfur-containing pesticides by the colorimetric sensor based on nano-mimetic enzyme.

### SUMMARY

Aiming at the shortcomings in the prior art, one of the objects of the disclosure is to provide a novel nano-quasi-peroxidase and a preparation method thereof. The second object of the disclosure is to provide a controllable preparation method of a nano-mimetic enzyme colorimetric sensor with simple preparation. The third object of the disclosure is to provide a highly sensitive method for detecting sulfur-containing pesticides in food, and the method has the advantages of simple operation, rapidity and sensitivity, and may realize rapid and accurate identification of sulfur-containing pesticides in complex substrates such as green tea, apples, Chinese cabbage and medlar.

In order to achieve the above object, the technical schemes adopted by the disclosure are as follows:

A novel nano-quasi-peroxidase is a bowl-shaped hollow structure with rough surface of gold-silver bimetallic nanocluster (Au—Ag NCs). The outer layer of the Au—Ag NCs is negatively charged. An average inner diameter of the Au—Ag NCs is 17.3±0.3 nanometers (nm), an average outer diameter of the Au—Ag NCs is 31.6±0.2 nm, aqueous dispersion of the Au—Ag NCs is purple, and λmax is 537±5 nm.

A preparation method of the novel nano-quasi-peroxidase takes trisodium citrate as a reducing agent and a stabilizing agent, and sequentially reacts with silver nitrate and chloroauric acid to synthesize hollow Au—Ag NCs, specifically including the following steps:
(1) heating and boiling a silver nitrate solution in dark, quickly adding a trisodium citrate aqueous solution while stirring (the optional stirring speed is 400 revolutions per minute (r/min)), uniformly mixing, stirring (the optional stirring speed is 200 r/min) and boiling in the dark for 1 hour (h); and
(2) after slowly adding (optionally adding at a constant speed of 30 drops per minute (min)) a chloroauric acid aqueous solution at 90 degrees Celsius (° C.) with stirring (the optional stirring speed is 200 r/min), continuing to stir at 90° C. in the dark for 45 min; and after completing a reaction, cooling a reaction system to room temperature, filtering an obtained solution, and then purifying to prepare the Au—Ag NCs.

Optionally, a molar ratio of silver nitrate, trisodium citrate and chloroauric acid is 0.05:1:0.015.

The Au—Ag NCs of the present disclosure may rapidly (may play a catalytic role within 30 seconds(s)) catalyze H₂O₂ to produce hydroxyl radical (OH) to oxidize 3,3′,5,5′-tetramethylbenzidine (TMB) to produce blue products in acidic environment, and exert peroxidase activity.

In the present disclosure, the novel nano-quasi-peroxidase is used as a colorimetric probe, hydrogen peroxide is used as an enzyme reaction substrate, and TMB is used as a chromogenic agent, and the colorimetric sensor is constructed by adding the three components in a specific order to a buffer solution system with appropriate pH. Optionally, the buffer solution is an acetic acid-sodium acetate buffer solution with a pH of 2.8≤pH≤4.6, and the optimal pH is 3.4.

A highly sensitive method for detecting four sulfur-containing pesticides in food is based on the electrostatic interaction between the negatively charged Au—Ag NCs and the positively charged sulfur-containing pesticides, exposing the inner Au and Ag elements of the mimetic enzyme material, and further combining with S in the pesticide molecules to form bonds, resulting in the collapse of the bowl-shaped hollow structure of Au—Ag NCs and the loss of mimetic enzyme activity, which may block the catalytic oxidation process and finally realize the determination the content of sulfur-containing pesticides in the system to be detected. The four sulfur-containing pesticides are thiophanate-methyl, cartap, dimethoate and temephos. The method specifically includes the following steps.
(1) drawing a standard curve: accurately weighing thiophanate-methyl standard, preparing a thiophanate-methyl standard stock solution with anhydrous methanol, and then diluting the thiophanate-methyl standard stock solution to different concentrations with ultrapure water to obtain a thiophanate-methyl linear solution, where the concentration ranges from 0.05 to 2 milligrams per liter (mg/L); adding the Au—Ag NCs, the thiophanate-methyl linear solution, hydrogen peroxide, an acetic acid-sodium acetate buffer solution with a pH of 2.8≤pH≤4.6 and a TMB solution into a reaction vessel in sequence, and mixing evenly; after reacting for 3 min, determining absorbance of the reaction system at 652 nm; and drawing the standard curve with concentration of the thiophanate-methyl as abscissa and the absorbance as ordinate;
drawing standard curves of the cartap, the dimethoate and the temephos by a same method;
where a cartap linear solution is prepared with the ultrapure water as solvent, and concentration ranges from 0.05 to 1 mg/L; a dimethoate linear solution is prepared by using absolute ethanol as solvent to prepare a stock solution, and then diluted with the ultrapure water, with concentration ranging from 0.01 to 2 mg/L; and a temephos linear solution is prepared by using the anhydrous methanol as solvent to prepare a stock solution, and then diluted with the ultrapure water, with concentration ranging from 0.01 to 1.5 mg/L; and
(2) detecting a sample: pretreating the sample to prepare a test solution; adding the Au—Ag NCs, the test solution, the hydrogen peroxide, the acetic acid-sodium acetate buffer solution with the pH of 2.8≤pH≤4.6 and the TMB solution into the reaction vessel in sequence, and mixing evenly; after reacting for 3 minutes, determining the absorbance of the reaction system at 652 nm; and substituting into a corresponding standard curve equation in the step (1), and calculating contents of the thiophanate-methyl, the cartap, the dimethoate and the temephos.

Among them, the pretreatment method in the step (2) includes the following steps: firstly crushing the solid sample, then adding pure water to juice or soak, filtering, and diluting the filtrate with pure water to colorless; and for liquid samples, shaking the liquid samples evenly first, then filtering the liquid samples, and diluting the filtrate with pure water to colorless.

Optionally, in the steps (1) and (2), aqueous dispersion of the Au—Ag NCs is added to the reaction vessel, and a mass ratio of the Au—Ag NCs to water is 2:1.

Optionally, in the steps (1) and (2), mass concentration of the hydrogen peroxide is 10%.

Optionally, in the steps (1) and (2), concentration of the acetic acid-sodium acetate buffer solution is 0.2 molar (M), and the pH is 3.4.

Optionally, in the steps (1) and (2), a dimethyl sulfoxide (DMSO) solution of TMB with concentration of 10 millimolar (mM) is added to the reaction vessel.

Optionally, in the steps (1) and (2), the volume ratio of Au—Ag NCs, a linear solution of the sulfur-containing pesticides/the test solution, the hydrogen peroxide, the acetic acid-sodium acetate buffer solution and TMB is 1:1:1:1.

Compared with the prior art, the disclosure has the following advantages and beneficial effects.

The Au—Ag NCs mimetic enzyme synthesized by the disclosure has good activity and short response time.

The methods of the disclosure may simultaneously detect four sulfur-containing pesticides, and has a wide application range.

The detection method of the disclosure has the advantages of high sensitivity, short detection time, simple operation, no need of expensive instruments and reagents, and low cost.

The detection method of the disclosure may directly judge whether the pesticide residue exceeds the standard with naked eyes.

### BRIEF DESCRIPTION OF THE DRAWINGS

FIG. 1 is a schematic diagram of the detection principle of the nano-mimetic enzyme colorimetric sensor of the present disclosure.

FIG. 2 shows the ultraviolet absorption spectrum of gold-silver bimetallic nanocluster (Au—Ag NCs) and its mimetic enzyme reaction in the nano-mimetic enzyme colorimetric sensor of the present disclosure, with wavelength as the abscissa and absorbance as the ordinate.

FIG. 3 shows the intensity of mimetic enzyme activity of Au—Ag NCs in buffer solutions with different pH values, with pH values on the abscissa and absorbance values on the ordinate.

FIG. 4A shows the variation trend of enzyme reaction rate with the concentration of H₂O₂.

FIG. 4B shows the inverse equation of the trend of enzyme reaction rate varying with the concentration of H₂O₂.

FIG. 4C shows the variation trend of enzyme reaction rate with 3,3′,5,5′-tetramethylbenzidine (TMB) concentration.

FIG. 4D shows the inverse equation of the trend of enzyme reaction rate varying with TMB concentration.

FIG. 5 is a fluorescence spectrum diagram of the Au—Ag NCs-H₂O₂-terephthalic acid (TA) system, with wavelength on the abscissa and fluorescence intensity on the ordinate.

FIG. 6 shows the Zeta potential results of Au—Ag NCs and four sulfur-containing pesticides, and the abscissa shows the potential difference, where TM is thiophanate-methyl, DT is dimethoate, CT is cartap, and TP is temephos.

FIG. 7A is a transmission electron microscope diagram of nano silver particles.

FIG. 7B is a partial enlarged view of the nano silver particles.

FIG. 7C is a transmission electron microscope diagram of Au—Ag NCs.

FIG. 7D is a partial enlarged view of the Au—Ag NCs.

FIG. 7E is a transmission electron microscope diagram of mixed solution of Au—Ag NCs and 10 weight percent (wt %) H₂O₂.

FIG. 7F is a partial enlarged view of the mixed solution of Au—Ag NCs and 10 wt % H₂O₂.

FIG. 8A is a transmission electron microscope diagram of Au—Ag NCs reacted with thiophanate-methyl in the nano-mimetic enzyme colorimetric sensor of the present disclosure.

FIG. 8B is a transmission electron microscope diagram of Au—Ag NCs reacted with cartap in the nano-mimetic enzyme colorimetric sensor of the present disclosure.

FIG. 8C is a transmission electron microscope diagram of Au—Ag NCs reacted with dimethoate in the nano-mimetic enzyme colorimetric sensor of the present disclosure.

FIG. 8D is a transmission electron microscope diagram of Au—Ag NCs reacted with temephos in the nano-mimetic enzyme colorimetric sensor of the present disclosure.

FIG. 9A is a visual effect diagram before and after the interaction between the mimetic enzyme detection system and thiophanate-methyl.

FIG. 9B is a visual effect diagram before and after the interaction between the mimetic enzyme detection system and cartap.

FIG. 9C is a visual effect diagram before and after the interaction between the mimetic enzyme detection system and dimethoate.

FIG. 9D is a visual effect diagram before and after the interaction between the mimetic enzyme detection system and temephos.

FIG. 10A shows the ultraviolet absorption spectrum of the standard curve after the sensor is constructed, where thiophanate-methyl with a known concentration is added, and the abscissa is wavelength and the ordinate is absorbance.

FIG. 10B shows the ultraviolet absorption spectrum of the standard curve after the sensor is constructed, where cartap with a known concentration is added, and the abscissa is wavelength and the ordinate is absorbance.

FIG. 10C shows the ultraviolet absorption spectrum of the standard curve after the sensor is constructed, where dimethoate with a known concentration is added, and the abscissa is wavelength and the ordinate is absorbance.

FIG. 10D shows the ultraviolet absorption spectrum of the standard curve after the sensor is constructed, where temephos with a known concentration is added, and the abscissa is wavelength and the ordinate is absorbance.

### DETAILED DESCRIPTION OF THE EMBODIMENTS

In the following, the applicant will further describe the present disclosure with reference to embodiments and drawings in the specification, but the scope of protection of the present disclosure is not limited to these embodiments. Those skilled in the art should understand that equivalent substitutions or corresponding improvements to the technical features of the disclosure still fall within the protection scope of the disclosure.

The experimental methods used in the following embodiments are all conventional methods unless otherwise specified. Unless otherwise specified, the materials and reagents used may be obtained from commercial sources. Among them, thiophanate-methyl standard is purchased from Sinopharm Group, with a purity of 99.8% and a specification of 25 milligrams (mg). The cartap standard is purchased from Sinopharm Group with a purity of 99.8% and a specification of 250 mg. The temephos standard is purchased from Sinopharm Group with a purity of 99.8% and a specification of 100 mg. The dimethoate standard is purchased from Sinopharm Group, with a purity of 99.8% and a specification of 100 mg.

### Embodiment 1: Preparation of Nano-Mimetic Enzyme Materials

A preparation method of novel nano-quasi-peroxidase, including the following steps:

In an oil bath, 50 milliliters (mL) of 0.1 millimolar (mM) silver nitrate aqueous solution is heated to boiling in dark. While maintaining vigorous stirring at 400 revolutions per minute (r/min) with an oil-bath magnetic stirrer, 1 mL of 0.1 molar (M) trisodium citrate aqueous solution is rapidly added. After homogeneous mixing, the stirring speed is adjusted to 200 r/min, and the reaction is allowed to proceed under light-protected boiling conditions for 1 hour (h), yielding a yellow transparent nano silver solution. Subsequently, under oil bath conditions at 90 degrees Celsius (° C.) with 200 r/min stirring, 1.5 mL of 1 mM chloroauric acid aqueous solution is added dropwise at a constant rate of 30 drops per minute (min). The light-protected reaction continues at 90° C. for 45 min to obtain a purple transparent solution. The resulting solution is filtered by a microporous membrane with a pore size of 0.22 micrometers (μm), followed by 24-hour purification using a cellulose dialysis bag (1000 Dalton (Da)). An aqueous dispersion of gold-silver bimetallic nanocluster (Au—Ag NCs) is obtained (the mass ratio of Au—Ag NCs to water is 2:1), exhibiting purple coloration with maximum ultraviolet absorption wavelength at 537 nanometers (nm). The final product is stored at 4° C. in a refrigerator for future use.

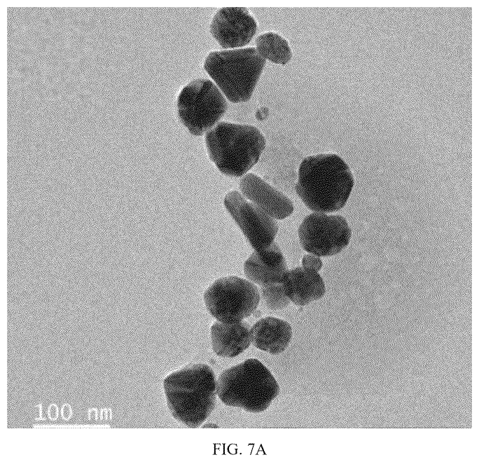

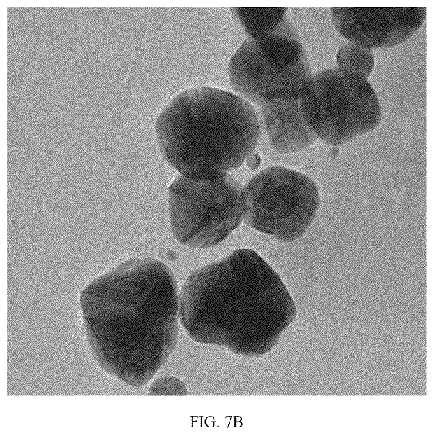

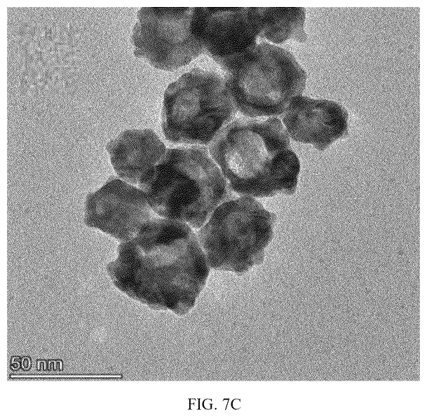

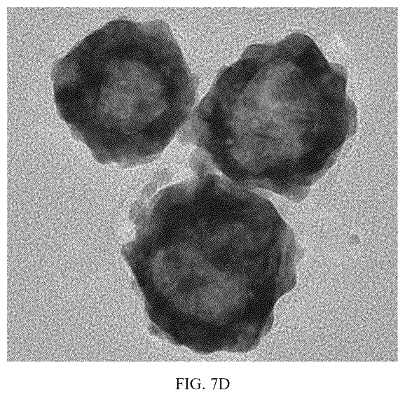

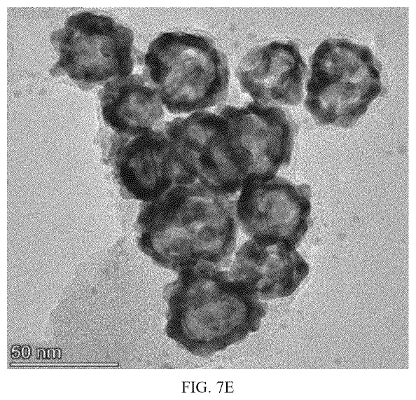

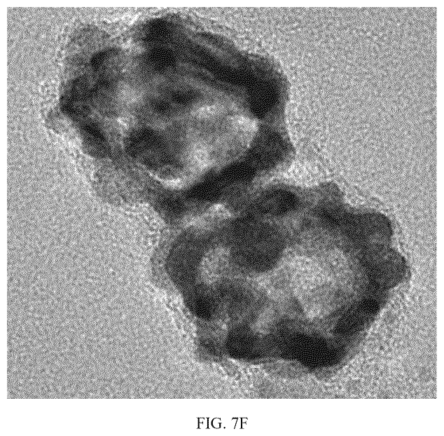

The prepared nano silver and Au—Ag NCs are scanned by a transmission electron microscope, and the obtained transmission electron microscopy (TEM) images are shown as FIG. 7A, FIG. 7B, FIG. 7C and FIG. 7D. TEM images show that the nano silver particles are nearly spherical with an average particle size of 44.9±0.3 nm. Au—Ag NCs is a bowl-shaped hollow structure, with an average inner diameter of 17.3±0.3 nm and an average outer diameter of 31.6±0.2 nm. The Au—Ag NCs has a rough surface and a large specific surface area.

### Embodiment 2: Investigation on the Mimetic Enzyme Activity of Au—Ag NCs

### 1. Nano-Mimetic Enzyme-Hydrogen Peroxide-3,3′,5,5′-Tetramethylbenzidine (TMB) System

### 1) Investigation of Ultraviolet Absorption Behavior

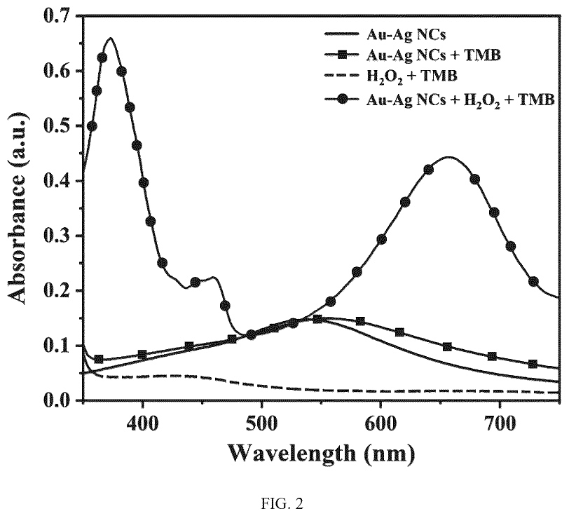

Au—Ag NCs of Embodiment 1, ultrapure water, 10 weight percent (wt %) hydrogen peroxide, 0.2 M acetic acid-sodium acetate buffer solution with pH 3.4, and 10 mM TMB are sequentially added into an Eppendorf (EP) tube, where the volume ratio of Au—Ag NCs, ultrapure water, hydrogen peroxide, buffer solution and TMB is 1:1:1:1:1. The mixture is thoroughly mixed and allowed to react for 3 min. The ultraviolet absorption spectrum is collected, as shown in FIG. 2 under the label “Au—Ag NCs+H₂O₂+TMB.”

The ultraviolet absorption spectra of “Au—Ag NCs” (Au—Ag NCs, ultrapure water, dimethyl sulfoxide (DMSO) and 0.2 M pH 3.4 acetic acid-sodium acetate buffer solution, with the volume ratio of 1:2:1:1), “Au—Ag NCs+TMB” (Au—Ag NCs, ultrapure water, 0.2 M pH 3.4 acetic acid-sodium acetate buffer solution and 10 mM TMB, with the volume ratio of 1:2:1:1) and “H₂O₂+TMB” (ultrapure water, 10 wt % hydrogen peroxide, 0.2 M pH 3.4 acetic acid-sodium acetate buffer solution and 10 mM TMB, with the volume ratio of 2:1:1:1) are collected by the same method.

In this embodiment and the following embodiments, Au—Ag NCs refers to the aqueous dispersion of Au—Ag NCs obtained during synthesis, and the concentration used is: the mass ratio of Au—Ag NCs to water is 2:1. TMB refers to DMSO solution of TMB.

As may be seen from FIG. 2, when Au—Ag NCs or H₂O₂ act alone, TMB does not produce the ultraviolet characteristic absorption peak of its oxidation products. Only when Au—Ag NCs interacts with H₂O₂, the characteristic absorption peaks of oxidized TMB appear at 652 nm and 370 nm. It is proved that Au—Ag NCs has quasi-peroxidase activity.

### 2) Investigation of Effect of pH

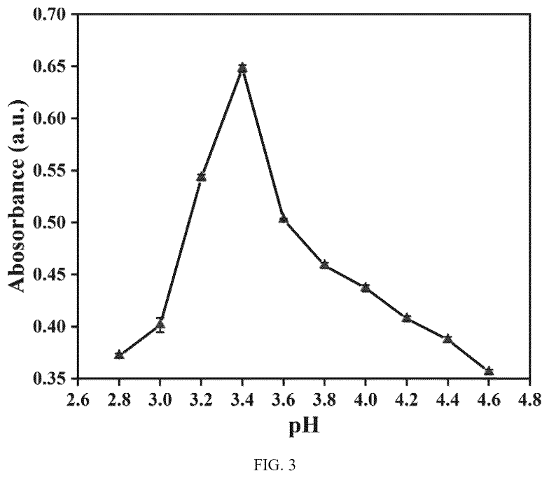

0.2 M acetic acid-sodium acetate buffer solutions with different pH values are prepared, and the ultraviolet absorption spectra of Au—Ag NCs+H₂O₂+TMB systems with different pH values are collected according to the method in 1) of this embodiment, and the absorbance value at 652 nm is recorded. The results are shown in FIG. 3. As may be seen from FIG. 3, when the pH of the buffer solution is 3.4, the peroxidase activity of Au—Ag NCs is the strongest.

### 3) Investigation on the Steady-State Kinetics of Enzyme Reaction

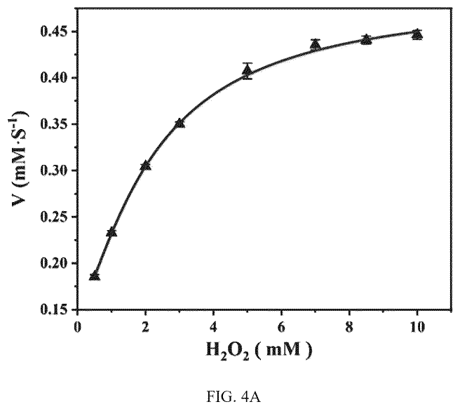

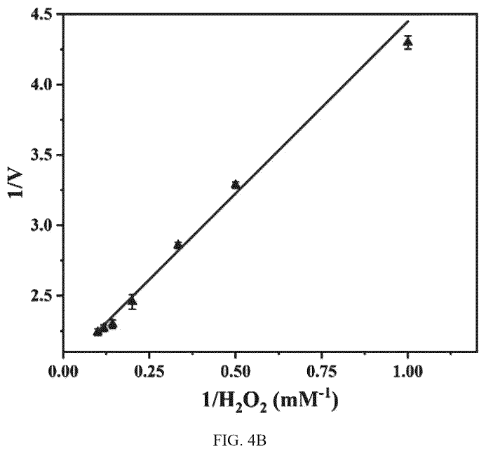

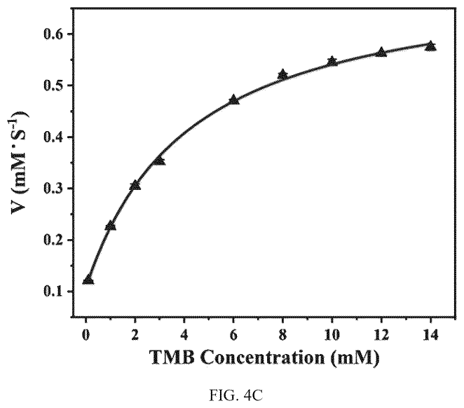

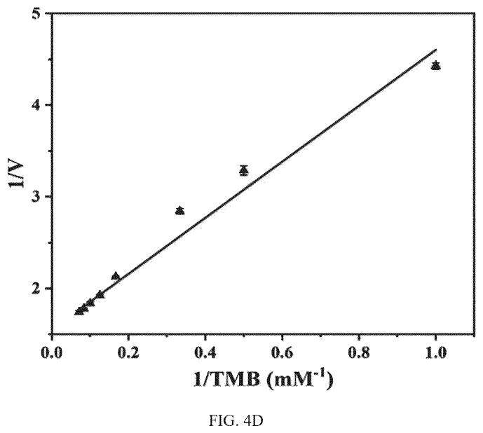

The concentration of TMB is fixed at 10 mM, and several H₂O₂ solutions with different concentrations are prepared. According to the sampling order of Au—Ag NCs, ultrapure water, H₂O₂ solution, 0.2 M pH 3.4 acetic acid-sodium acetate buffer solution and TMB (the volume ratio is 1:1:1:1:1), the absorbance at 652 nm at the same reaction time is collected, and the reaction rate is calculated. On the contrary, when the concentration of H₂O₂ is fixed at 10 wt %, multiple TMB solutions with different concentrations are prepared, and the absorbance at 652 nm is collected by the same method, and the reaction rate is calculated. The Michaelis curve is drawn, and the result is shown in FIG. 4A, FIG. 4B, FIG. 4C and FIG. 4D.

According to Michaelis equation, when Au—Ag NCs undergoes mimetic enzyme reaction, Kₘ=899.3 micromolar (μM) and Vₘₐₓ=0.446 millimolar per second (mM·S⁻¹) when TMB is used as substrate; when H₂O₂ is used as substrate, Kₘ=1672.3 (μM) and Vₘₐₓ=0.575 (mM·S⁻¹). Compared with other reported quasi-peroxidase, the activity is stronger (Kₘ is the kinetic constant of enzyme reaction, Vₘₐₓ is the maximum reaction rate of enzyme; the smaller the Kₘ, the greater the Vₘₐₓ, the better the mimetic enzyme activity).

TABLE 1
Comparison of parameters between Au—Ag NCs of the present
disclosure and other quasi-peroxidase Michaelis equation

| Mimetic enzyme | | Kₘ | Vₘₐₓ | |
|---|---|---|---|---|
| materials | Substrate | (μM) | (mM · S⁻¹) | References |
| Au—Ag NCs | TMB | 899.3 | 0.446 | The present |
| | | | | disclosure |
| Au—Ag NCs | H₂O₂ | 1672.3 | 0.575 | The present |
| | | | | disclosure |
| BSA-Au NCs | TMB | 3590 | 0.861 × 10⁻⁵ | [1] |
| BSA-Au NCs | H₂O₂ | 16710 | 1.3 × 10⁻⁵ | [1] |
| HRP | TMB | 434 | 0.201 | [2] |
| HRP | H₂O₂ | 3700 | 0.334 | [2] |

### 2. Nano-Mimetic Enzyme-Hydrogen Peroxide-Terephthalic Acid (TA) System

The Au—Ag NCs of Embodiment 1 is mixed with 10 wt % hydrogen peroxide, then 0.2 M pH 3.4 acetic acid-sodium acetate buffer solution is added to react for 3-5 min, then 5 mM TA solution (solvent is 0.1 M NaOH) is added, and finally 0.2 mM pH 5.0 phosphoric acid buffer solution is added. Among them, the volume ratio of Au—Ag NCs, hydrogen peroxide, acetic acid-sodium acetate buffer solution, TA solution and phosphoric acid buffer solution is 1:1:1:1:1. The mixture is mixed evenly, and the fluorescence spectrum of the solution is collected after 20 min of reaction. The acquisition conditions of fluorescence spectrum are Eₓ=316 nm, Eₘ=300-600 nm, voltage=400 volts (V), and slit width=10 nm.

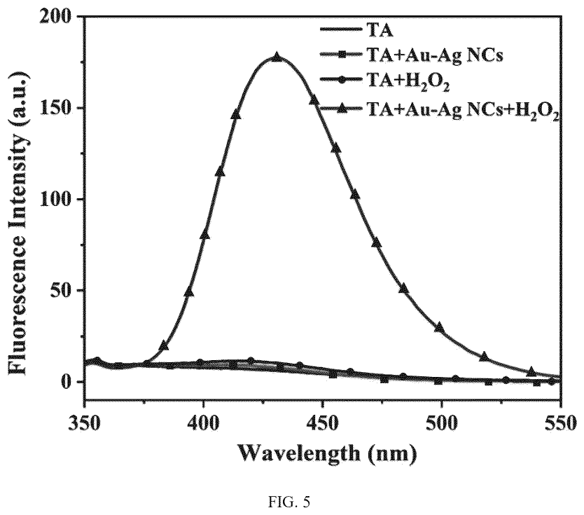

The fluorescence spectra of “TA” (ultrapure water, 0.2 M pH 3.4 acetic acid-sodium acetate buffer solution, 5 mM TA solution and 0.2 mM pH 5.0 phosphoric acid buffer solution, with the volume ratio of 2:1:1:1), “TA+Au—Ag NCs” (Au—Ag NCs, ultrapure water, 0.2 M pH 3.4 acetic acid-sodium acetate buffer solution, 5 mM TA solution and 0.2 mM pH 5.0 phosphoric acid buffer solution, with the volume ratio of 1:1:1:1:1) and “TA+H₂O₂” (ultrapure water, 10 wt % hydrogen peroxide, 0.2 M pH 3.4 acetic acid-sodium acetate buffer solution, 5 mM TA solution and 0.2 mM pH 5.0 phosphoric acid buffer solution, with the volume ratio of 1:1:1:1:1) are collected in the same way, and the results are shown in FIG. 5.

As may be seen from FIG. 5, the products obtained by the reaction of purple Au—Ag NCs nano-mimetic enzyme materials with H₂O₂ in acidic environment may react with non-fluorescent TA, resulting in fluorescence. Under the condition of Eₓ=316 nm, the fluorescence characteristic peak of 2-hydroxyterephthalic acid is detected at Eₘ=425 nm by fluorescence spectrophotometer, which confirms the generation of hydroxyl radical (·OH). The principle of this reaction is that the negative charge on the outer layer of Au—Ag NCs undergoes electron transfer with H₂O₂ to generate ·OH, which may react with TA to generate stable and unique 2-hydroxyterephthalic acid with strong blue fluorescence.

### Embodiment 3: Establishment of Analytical Method

Thiophanate-methyl standard is accurately weighed, and a 1000 milligrams per liter (mg/L) thiophanate-methyl standard stock solution is prepared using anhydrous methanol as the solvent. The thiophanate-methyl standard stock solution is then diluted with ultrapure water to concentrations of 10, 5, 3, 1, 0.8, 0.5, and 0.1 mg/L, respectively.

Cartap standard is accurately weighed, and a 1000 mg/L cartap standard stock solution is prepared using ultrapure water as the solvent. The cartap standard stock solution is then diluted with ultrapure water to concentrations of 10, 5, 3, 1, 0.8, 0.5, and 0.1 mg/L, respectively.

Dimethoate standard is accurately weighed, and a 1000 mg/L dimethoate standard stock solution is prepared using absolute ethanol as the solvent. The dimethoate standard stock solution is then diluted with ultrapure water to concentrations of 10, 5, 3, 1, 0.8, 0.5, and 0.1 mg/L, respectively.

temephos standard is accurately weighed, and a 1000 mg/L temephos standard stock solution is prepared using anhydrous methanol as the solvent. The temephos standard stock solution is then diluted with ultrapure water to concentrations of 10, 5, 3, 1, 0.8, 0.5, and 0.1 mg/L, respectively.

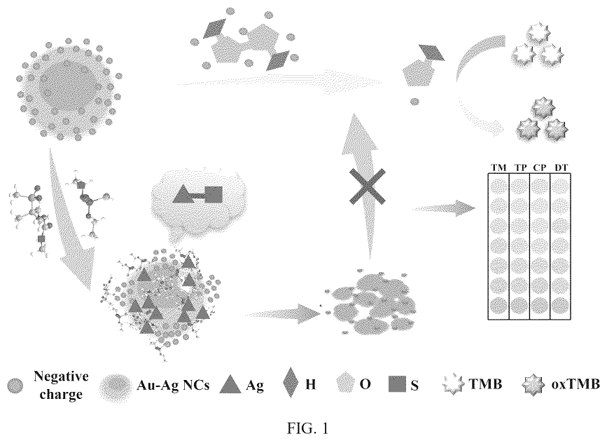

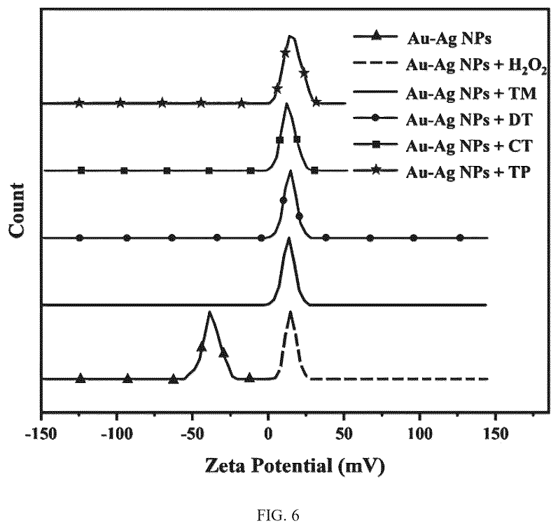

The Au—Ag NCs of Embodiment 1 are mixed with 10 wt % hydrogen peroxide, 1 mg/L thiophanate-methyl, 1 mg/L temephos, 1 mg/L dimethoate and 1 mg/L cartap in a volume ratio of 1:1, and the potential difference of each mixed solution and Au—Ag NCs is collected by Zeta potentiometer. The results are shown in FIG. 6. As may be seen from FIG. 6, Au—Ag NCs is negatively charged, four sulfur-containing pesticides are positively charged, and electrostatic interaction may occur between Au—Ag NCs and four sulfur-containing pesticides, so there are favorable reaction conditions. This conclusion corresponds to the reaction diagram in FIG. 1.

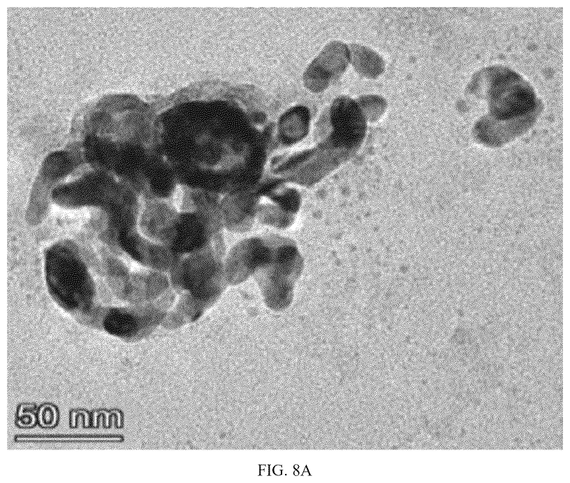

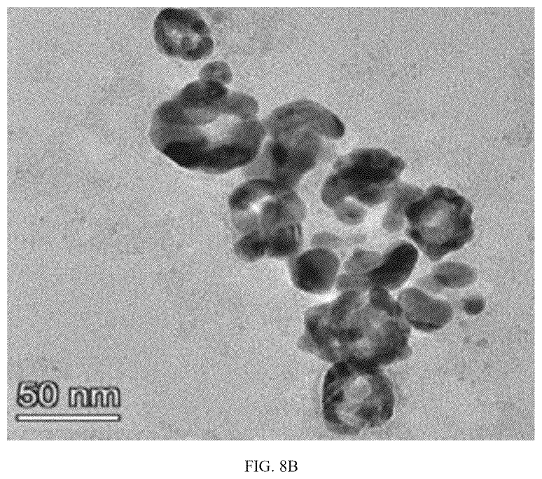

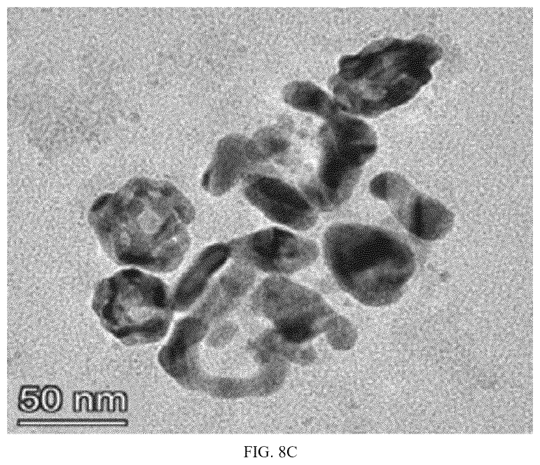

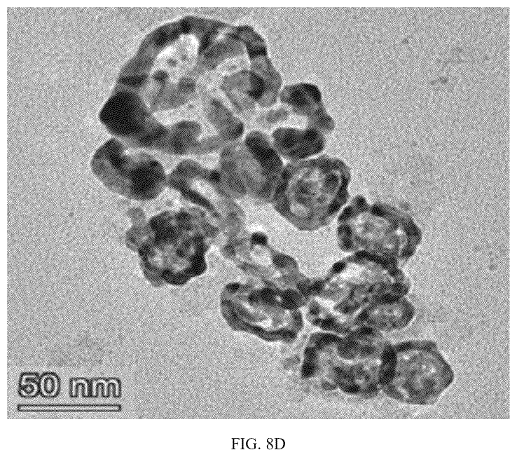

The Au—Ag NCs of Embodiment 1 are mixed with 10 wt % hydrogen peroxide, 10 mg/L thiophanate-methyl, 10 mg/L temephos, 10 mg/L dimethoate and 10 mg/L cartap in a volume ratio of 1:1, and the microscopic images of each mixed solution are taken by transmission electron microscope. The results are shown in FIG. 7E, FIG. 7F, FIG. 8A, FIG. 8B, FIG. 8C and FIG. 8D. From FIG. 7E and FIG. 7F, it may be seen that the hollow diameter of Au—Ag NCs increases after the mimetic enzyme reaction. As may be seen from FIG. 8A, FIG. 8B, FIG. 8C and FIG. 8D, the bowl-shaped hollow structure disintegrates after Au—Ag NCs reacts with four sulfur-containing pesticides. This conclusion corresponds to the reaction diagram in FIG. 1.

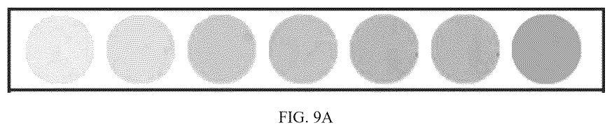

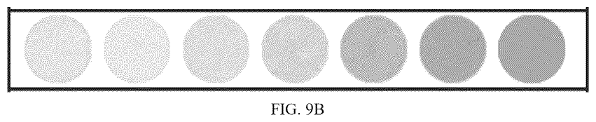

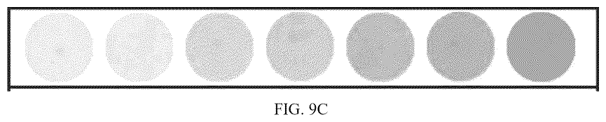

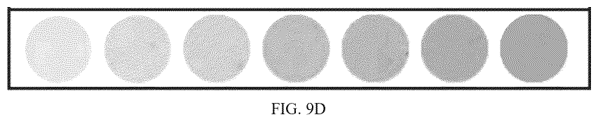

The following pesticide standard solutions are taken respectively: thiophanate-methyl, dimethoate, temephos and cartap with the concentrations of 5, 3, 1, 0.8, 0.5, 0.1 and 0 mg/L. The solutions are mixed in the following order: Au—Ag NCs, pesticide standard solution, 10 wt % hydrogen peroxide, 0.2 M pH 3.4 acetic acid-sodium acetate buffer solution, and 10 mM TMB, with a volume ratio of 1:1:1:1:1. The mixture is thoroughly mixed and allowed to react for 3 min. The camera is used to shoot the colorimetric effect diagram, and the result is shown in FIG. 9A, FIG. 9B, FIG. 9C and FIG. 9D (from left to right, the concentration of pesticide standard solution decreases gradually). As may be seen from FIG. 9A, FIG. 9B, FIG. 9C and FIG. 9D, in the colorimetric sensing system, the color changes from shallow to deep according to the concentration of pesticide standard solution from high to low, which may be clearly observed by naked eyes, and the effect of the colorimetric sensing method for identifying sulfur-containing pesticides constructed in this disclosure is satisfactory.

As shown in FIG. 1, the reaction principle is as follows: due to the electrostatic attraction between the outer electrons of Au—Ag NCs and four sulfur-containing pesticides, Au and Ag elements in the inner layer of Au—Ag NCs are exposed. There are lone electron pairs in S in four sulfur-containing pesticides, which may combine with Au and Ag elements in the inner layer of Au—Ag NCs to form bonds. After sulfur-containing pesticides combine with Au and Ag elements in the inner layer of Au—Ag NCs to form bonds, the structure of Au—Ag NCs is destroyed and the activity of mimetic enzyme is blocked.

### Embodiment 4: Validation of Analytical Method

### (1) Linearity, Quantitative Limit and Detection Limit

The linear solution of sulfur-containing pesticides is prepared: The thiophanate-methyl standard stock solution from Embodiment 3 is taken. The stock solution is diluted with ultrapure water to concentrations of 2, 1.5, 1, 0.7, 0.5, 0.3, 0.1, and 0.05 mg/L, respectively. The cartap standard stock solution from Embodiment 3 is taken. The stock solution is diluted with ultrapure water to concentrations of 1, 0.8, 0.5, 0.3, 0.1, 0.08, and 0.05 mg/L, respectively. The dimethoate standard stock solution from Embodiment 3 is taken. The stock solution is diluted with ultrapure water to concentrations of 2, 1.5, 1, 0.8, 0.5, 0.3, 0.1, 0.05, and 0.01 mg/L, respectively. The temephos standard stock solution from Embodiment 3 is taken. The stock solution is diluted with ultrapure water to concentrations of 1.5, 1, 0.8, 0.5, 0.3, 0.1, 0.05, and 0.01 mg/L, respectively.

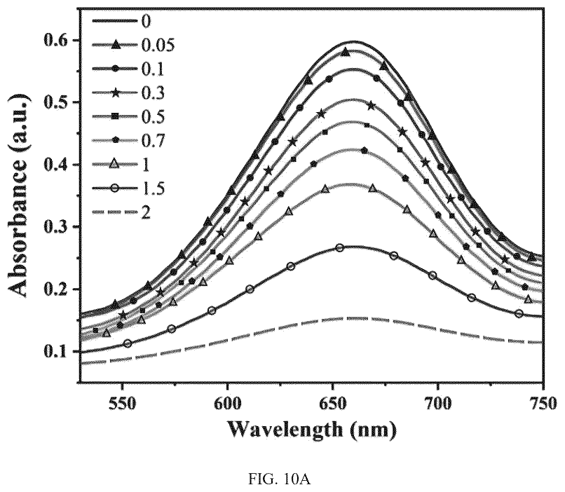

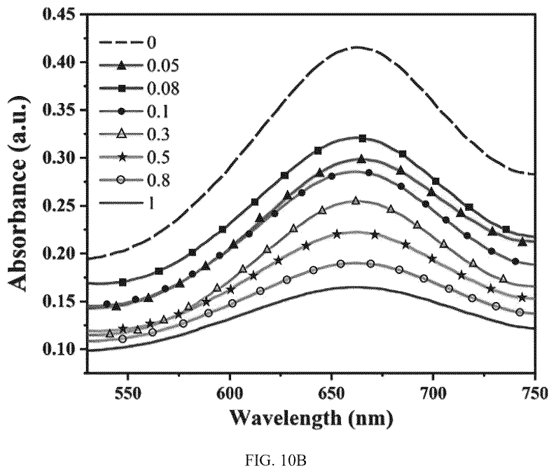

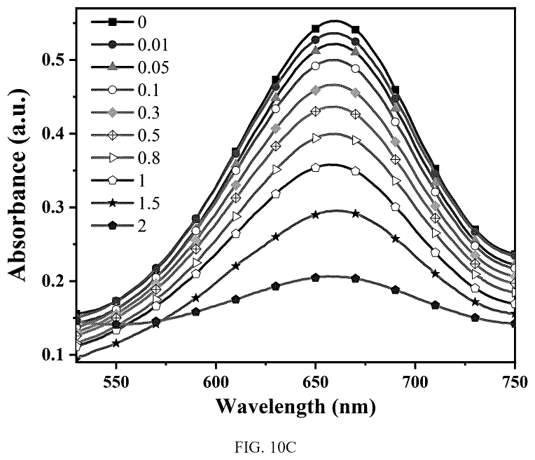

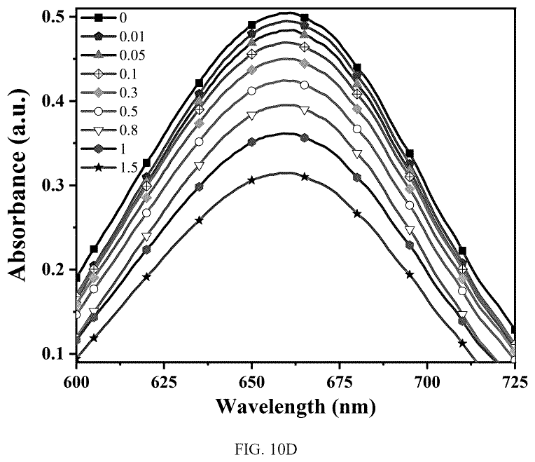

Au—Ag NCs of Embodiment 1, linear solution of sulfur-containing pesticides, 10 wt % hydrogen peroxide, 0.2 M pH 3.4 acetic acid-sodium acetate buffer solution and 10 mM TMB are sequentially added into an EP tube, where the volume ratio of Au—Ag NCs, linear solution, hydrogen peroxide, buffer solution and TMB is 1:1:1:1. The mixture is thoroughly mixed and allowed to react for 3 min. The ultraviolet absorption spectrum of the reaction system is then collected (FIG. 10A, FIG. 10B, FIG. 10C and FIG. 10D), with the absorbance at 652 nm being recorded. The standard curve is drawn with the concentration of linear solution as abscissa and absorbance as ordinate. The standard curve equation and linear correlation coefficient of each sulfur-containing pesticide are shown in Table 2 below.

According to the formula: LOQ=(10σ)/k, the quantitative limit of the method is calculated, where σ is the standard deviation of absorbance of the blank control measured for 10 times, and k is the slope of the standard curve. The calculated quantitative limits of four sulfur-containing pesticides are shown in Table 2 below.

According to the formula: LOD=(3σ)/k, the detection limit of the method is calculated, where σ is the standard deviation of the absorbance of the blank control measured for 10 times, and k is the slope of the standard curve. The calculated detection limits of four sulfur-containing pesticides are shown in Table 2 below.

TABLE 2
Linear equation, correlation coefficient, quantitative limit
and detection limit of four sulfur-containing pesticides

| | | | Quantitative | Detec- |
|---|---|---|---|---|
| | | | limit (parts | tion |
| | | | per million | limit |
| Pesticides | Linear equation | R² | (ppm)) | (ppm) |
| Thiophanate- | A = −0.21932C + 0.57907 | 0.994 | 0.076 | 0.023 |
| methyl | | | | |
| Cartap | A = −0.13405C + 0.29760 | 0.997 | 0.089 | 0.027 |
| temephos | A = −0.11603C + 0.48746 | 0.996 | 0.044 | 0.013 |
| Dimethoate | A = −0.15769C + 0.52395 | 0.995 | 0.054 | 0.016 |

### (2) Accuracy

Samples of green tea, apples, Chinese cabbage and medlar are pulverized, respectively, and a proper amount of ultrapure water is added for juicing or soaking, the mixtures are filtered, and the filtrate is diluted to colorless with ultrapure water.

The sulfur-containing pesticide standard stock solution from Embodiment 3 is taken and diluted with the aforementioned food extract solutions to three concentration levels: high (1 mg/L), medium (0.5 mg/L), and low (0.1 mg/L), to obtain the recovery rate test solutions. According to the method in (1) of this embodiment, a reaction system is constructed (three groups of reaction systems are set in parallel), and the absorbance at 652 nm is measured. The absorbance value is substituted into the standard curve equation in (1) to obtain the test concentration of sulfur-containing pesticides. According to the formula: recovery rate of standard addition=(test concentration/sample concentration)*100%, the recovery rates of four sulfur-containing pesticides in actual samples are calculated. The results are shown in Table 3 below.

TABLE 3

| | | | | | Relative |
|---|---|---|---|---|---|
| | | Sample | Test | | standard |
| | | concen- | concen- | | deviation |
| | Actual | tration | tration | Recovery | (RSD) (%, |
| Pesticides | samples | (mg/L) | (mg/L) | rate (%) | n = 3) |
| temephos | Chinese | 0.1 | 0.11 | 106.24 | 2.04 |
| | cabbage | 0.5 | 0.53 | 106.51 | 0.61 |
| | | 1 | 1.05 | 104.59 | 1.79 |
| | Green | 0.1 | 0.11 | 106.52 | 3.53 |
| | tea | 0.5 | 0.54 | 107.66 | 0.58 |
| | | 1 | 1.04 | 103.67 | 4.30 |
| | Medlar | 0.1 | 0.10 | 104.14 | 3.28 |
| | | 0.5 | 0.53 | 105.48 | 2.50 |
| | | 1 | 1.05 | 105.46 | 3.69 |
| | Apples | 0.1 | 0.10 | 103.65 | 3.15 |
| | | 0.5 | 0.51 | 101.11 | 1.19 |
| | | 1 | 0.96 | 96.32 | 0.50 |
| Thiophanate- | Chinese | 0.1 | 0.11 | 107.03 | 4.21 |
| methyl | cabbage | 0.5 | 0.56 | 111.65 | 3.01 |
| | | 1 | 0.98 | 97.56 | 1.71 |
| | Green | 0.1 | 0.11 | 108.68 | 2.53 |
| | tea | 0.5 | 0.53 | 105.39 | 1.91 |
| | | 1 | 0.97 | 97.06 | 3.59 |
| | Medlar | 0.1 | 0.11 | 108.23 | 4.94 |
| | | 0.5 | 0.51 | 102.20 | 0.54 |
| | | 1 | 0.98 | 97.90 | 1.47 |
| | Apples | 0.1 | 0.11 | 107.04 | 4.75 |
| | | 0.5 | 0.52 | 103.20 | 1.10 |
| | | 1 | 0.97 | 97.06 | 0.68 |
| Dimethoate | Chinese | 0.1 | 0.11 | 105.16 | 4.44 |
| | cabbage | 0.5 | 0.53 | 105.29 | 0.30 |
| | | 1 | 1.13 | 113.25 | 3.67 |
| | Green | 0.1 | 0.11 | 108.12 | 1.76 |
| | tea | 0.5 | 0.55 | 110.83 | 4.99 |
| | | 1 | 1.01 | 101.37 | 4.17 |
| | Medlar | 0.1 | 0.11 | 109.39 | 3.23 |
| | | 0.5 | 0.57 | 113.75 | 3.97 |
| | | 1 | 1.04 | 103.97 | 1.90 |
| | Apples | 0.1 | 0.11 | 105.63 | 2.22 |
| | | 0.5 | 0.56 | 112.10 | 1.84 |
| | | 1 | 1.06 | 106.02 | 1.26 |
| Cartap | Chinese | 0.1 | 0.11 | 112.89 | 2.32 |
| | cabbage | 0.5 | 0.52 | 103.44 | 0.79 |
| | | 1 | 1.06 | 105.78 | 1.94 |
| | Green | 0.1 | 0.11 | 105.68 | 2.67 |
| | tea | 0.5 | 0.50 | 99.76 | 3.35 |
| | | 1 | 0.98 | 97.77 | 3.35 |
| | Medlar | 0.1 | 0.10 | 104.94 | 4.17 |
| | | 0.5 | 0.52 | 104.24 | 4.44 |
| | | 1 | 0.98 | 97.97 | 3.43 |
| | Apples | 0.1 | 0.10 | 102.45 | 2.34 |
| | | 0.5 | 0.54 | 107.72 | 4.58 |
| | | 1 | 1.01 | 100.79 | 3.38 |

As may be seen from Table 3, the recovery rate of standard addition of four sulfur-containing pesticides in apples, medlar, green tea and Chinese cabbage at three concentration levels are all between 95% and 115%, and the RSD is less than 5%, which proves that the colorimetric sensor of the disclosure has satisfactory detection effect in actual samples.

### REFERENCES

[1] Hu, L.; Liao, H.; Feng, L.; Wang, M.; Fu, W. Accelerating the Peroxidase-Like Activity of Gold Nanoclusters at Neutral pH for Colorimetric Detection of Heparin and Heparinase Activity. Anal. Chem. 2018, 90, 6247-6252.
[2] HUANG X, NAN Z. Porous 2D FeS₂ nanosheets as a peroxidase mimic for rapid determination of H₂O₂ [J]. Talanta, 2020, 216.

What is claimed is:

1. A preparation method of nano-quasi-peroxidase, wherein the nano-quasi-peroxidase is a gold-silver bimetallic nanocluster Au—Ag NCs, and the Au—Ag NCs has a bowl-shaped hollow structure with a rough surface and a negatively charged outer layer;
wherein the preparation method comprises following steps:
(1) heating and boiling a silver nitrate solution in dark, quickly adding a trisodium citrate aqueous solution while stirring, uniformly mixing, stirring and boiling in the dark for 1 h; and
(2) after slowly adding a chloroauric acid aqueous solution at 90° C. with stirring, continuing to stir at 90° C. in the dark for 45 min; and after completing a reaction, cooling a reaction system to room temperature, filtering an obtained solution, and then purifying to prepare the Au—Ag NCs.

2. The preparation method according to claim 1, wherein an average inner diameter of the Au—Ag NCs is 17.3±0.3 nm, an average outer diameter of the Au—Ag NCs is 31.6±0.2 nm, aqueous dispersion of the Au—Ag NCs is purple, and λmax is 537±5 nm.

3. The preparation method according to claim 1, wherein in the step (1), a stirring speed is 400 r/min when adding the trisodium citrate aqueous solution, and the stirring speed is 200 r/min when boiling in the dark; and
in the step (2), 1-2 min is taken to finish adding the chloroauric acid aqueous solution, and the stirring speed when adding the chloroauric acid aqueous solution is 200 r/min.

4. The preparation method according to claim 1, wherein a molar ratio of the silver nitrate, the trisodium citrate and the chloroauric acid is 0.05:1:0.015.

5. A colorimetric sensor of the nano-quasi-peroxidase prepared by the preparation method according to claim 1, wherein the nano-quasi-peroxidase is used as a colorimetric probe, hydrogen peroxide is used as an enzyme reaction substrate, 3,3′,5,5′-tetramethylbenzidine is used as a chromogenic agent, and an acetic acid-sodium acetate buffer solution with a pH of 2.8≤pH≤4.6 is used as a buffer system.

6. A method for quantitatively detecting four sulfur-containing pesticides in food by the nano-quasi-peroxidase prepared based on the preparation method according to claim 1, wherein the four sulfur-containing pesticides are thiophanate-methyl, cartap, dimethoate and temephos;
wherein the method for quantitatively detecting the four sulfur-containing pesticides in the food comprises following steps:
(1) drawing a standard curve: accurately weighing thiophanate-methyl standard, preparing a thiophanate-methyl standard stock solution with anhydrous methanol, and then diluting the thiophanate-methyl standard stock solution to different concentrations with ultrapure water to obtain a thiophanate-methyl linear solution, wherein the concentrations range from 0.05 to 2 mg/L; adding the Au—Ag NCs, the thiophanate-methyl linear solution, hydrogen peroxide, an acetic acid-sodium acetate buffer solution with a pH of 2.8≤pH≤4.6 and a 3,3′,5,5′-tetramethylbenzidine (TMB) solution into a reaction vessel in sequence, and mixing evenly; after reacting for 3 minutes, determining absorbance of the reaction system at 652 nm; and drawing the standard curve with concentration of the thiophanate-methyl as abscissa and the absorbance as ordinate;
drawing standard curves of the cartap, the dimethoate and the temephos by a same method;
wherein a cartap linear solution is prepared with the ultrapure water as solvent, and concentration ranges from 0.05 to 1 mg/L; a dimethoate linear solution is prepared by using absolute ethanol as solvent to prepare a first stock solution, and then diluted with the ultrapure water, with concentration ranging from 0.01 to 2 mg/L; and a temephos linear solution is prepared by using the anhydrous methanol as solvent to prepare a second stock solution, and then diluted with the ultrapure water, with concentration ranging from 0.01 to 1.5 mg/L; and
(2) detecting a sample: pretreating the sample to prepare a test solution; adding the Au—Ag NCs, the test solution, the hydrogen peroxide, the acetic acid-sodium acetate buffer solution with the pH of 2.8≤pH≤4.6 and the TMB solution into the reaction vessel in sequence, and mixing evenly; after reacting for 3 minutes, determining the absorbance of the reaction system at 652 nm; and substituting into a corresponding standard curve equation in the step (1), and calculating contents of the thiophanate-methyl, the cartap, the dimethoate and the temephos.

7. The method according to claim 6, wherein in the steps (1) and (2), aqueous dispersion of the Au—Ag NCs is added to the reaction vessel, and a mass ratio of the Au—Ag NCs to water is 2:1;
in the steps (1) and (2), mass concentration of the hydrogen peroxide is 10%;
in the steps (1) and (2), a concentration of the acetic acid-sodium acetate buffer solution is 0.2 M, and the pH is 3.4; and
in the steps (1) and (2), a dimethyl sulfoxide (DMSO) solution of TMB with concentration of 10 mM is added to the reaction vessel.

8. The method according to claim 7, wherein in the steps (1) and (2), a volume ratio of the aqueous dispersion of the Au—Ag NCs, a linear solution of the sulfur-containing pesticides/the test solution, the hydrogen peroxide, the acetic acid-sodium acetate buffer solution and the DMSO solution of the TMB is 1:1:1:1:1.
